(19) United States
(12) Patent Application Publication
(10) Pub. No.: US 2018/0326162 A1
(43) Pub. Date: Nov. 15, 2018

(54) System, Device, and Method to Adapt Electrodes to the Skin to Reduce Injection Pain

(71) Applicant: Pourang Bral, Passaic, NJ (US)

(72) Inventor: Pourang Bral, Passaic, NJ (US)

(21) Appl. No.: 15/977,540

(22) Filed: May 11, 2018

Related U.S. Application Data
(60) Provisional application No. 62/602,944, filed on May 11, 2017.
(60) Provisional application No. 62/602,943, filed on May 11, 2017.

(51) Int. Cl. A61M 5/42; A61N 1/04; A61N 1/02; A61N 1/36

(52) U.S. Cl. CPC A61M 5/422; A61N 1/0456; A61N 1/0472; A61M 2205/054; A61N 1/36021; A61N 1/3603; A61N 1/025

(57) ABSTRACT

Systems, devices and methods for reducing injection pain by ensuring that one or more skin stimulators are within a predefined range of operating distances from the patient's skin. In some examples, a sensor detects a distance between the patient's skin and a stimulator. A practitioner can be alerted when the stimulator is outside the predefined range. The predefined range can depend on several factors.

A device to reduce or remove the injection pain is disclosed in patent application U.S. Ser. No. 12/590,658, U.S. Ser. No. 13/134,013, U.S. Ser. No. 13/550,826, U.S. Ser. No. 14/140,518, U.S. Ser. No. 12/927,136, and the provisional application U.S. 62/389,529, among others, that uses electrodes to apply electricity to the skin of an injection recipient prior to an injection in order to reduce or remove the pain of an injection. It is desirable to use this device for reducing the injection pain in various body parts. Different recipient body parts have different contours. Furthermore, the same body part on different recipients has different contours. It is necessary that the device electrodes make proper contact with the skin to adequately supply electricity to the skin. The electrodes used in the device should be adaptable to various contours and thicknesses of different body parts of a variety of people.

### BACKGROUND

### Summary

In order to adapt the electrodes to various body contours, one may increase the pressure of the electrodes on the skin. However, this undesirably causes the tissue to bulge around the electrodes 2. Bulging tissue can be especially problematic when the device that provides the electrical stimulation to the skin includes one or more other physical stimulations to the skin, such as tapping on the skin, rubbing the skin, or blowing gas, etc, where the stimulator mechanism is preferably positioned at a certain distance from the skin. Bulging tissue can unpredictably alter or distort the distance between the stimulating mechanism and the skin, thereby reducing the effectiveness of the pain relieving device.

Thus, there is a need for improved adaptability of pain relieving electrodes to the skin of different patients.

In general terms, the present disclosure is directed to a device and a method to effectively supply electricity to the skin at an injection site and physically stimulate the injection site prior to an injection in order to reduce the pain of an injection.

According to certain aspects of the present disclosure, the device and method are adapted to at least substantially provide a consistent, predefined distance, or at least a predefined range of distances, between the skin of the recipient and the mechanism of the device that physically stimulates the skin. In some examples, the predefined distance or predefined range of distances is selected to optimize the injection-pain reduction qualities of the device. In some examples, the optimization depends on one or more factors, including but not limited to: the number of stimuli provided by the device, the type(s) of stimuli provided by the device, the intensity of the stimuli provided by the device, the relative timing of application of the different stimuli provided by the device to the patient's skin, the location of the injection on the person, qualities of the skin and other tissues at and around the location of the injection, the type of needle or other injection device, the depth of the injection, the duration of the injection, and so forth.

### BRIEF DESCRIPTION OF THE DRAWINGS

The characteristics of the present disclosure will be better understood by reading the following description, given purely by way of example, and with reference to the accompanying drawings, in which:

FIG. 1 is a schematic representation of an example device for reducing pain of an injection in accordance with the present disclosure, the device being shown in proximity to a patient's skin.

FIG. 2 shows a sensor that signals to a processor when a stimulator, included in a device that is moving towards the skin around the device, reaches a certain preset distance from the skin near the injection site that is being anesthetized.

FIG. 3 shows a sensor that is a laser.

FIG. 4 shows a sensor positioned a distance away from the injection site that sends a signal when the sensor touches the skin near the injection site.

FIG. 5 exhibits an embodiment of the disclosure with two electrodes held at one end by a device.

FIG. 6 shows two electrodes held to a device to reduce pain of skin penetration by supports that flex or move, for example in a rotational movement, when the device is pressed against the skin to allow the stimulator approach closer to the skin at the injection site.

FIG. 7 illustrates an electrode held to a support by a swivel that helps the electrode to better adapt to the contour of the skin.

FIG. 8 shows the side view of a device to reduce pain of skin invasion being pressed towards the skin. The device holds a support that bears an electrode. The support can be pushed into the device by pressure.

FIG. 9 Shows a device to reduce pain of skin penetration including one electrode touching the skin. The device can be so tilted as to vary the distance between the included stimulator and the skin.

FIG. 10a exhibits a sample of electrodes that are brought together when not in use to avoid drying of the electrolytes that are usually placed on the electrodes in order to facilitate transmission of electricity from electrodes to the skin.

FIG. 10b shows electrodes folding on each other.

FIG. 11 illustrates another version of electrodes that fold over themselves or other electrode to protect the electrolyte on them.

FIG. 12 shows another version of an electrode that folds over itself or another electrode to protect the electrolyte on it or them.

FIG. 13 shows a lateral view of a device to reduce an injection pain.

FIG. 14 shows an embodiment of the present disclosure tilted on the skin.

FIG. 15 illustrates an embodiment that prevents the electrolyte from drying and the stimulator from contamination.

FIG. 16 shows a hinge having one component that can slide and rotate against another.

### DETAILED DESCRIPTION

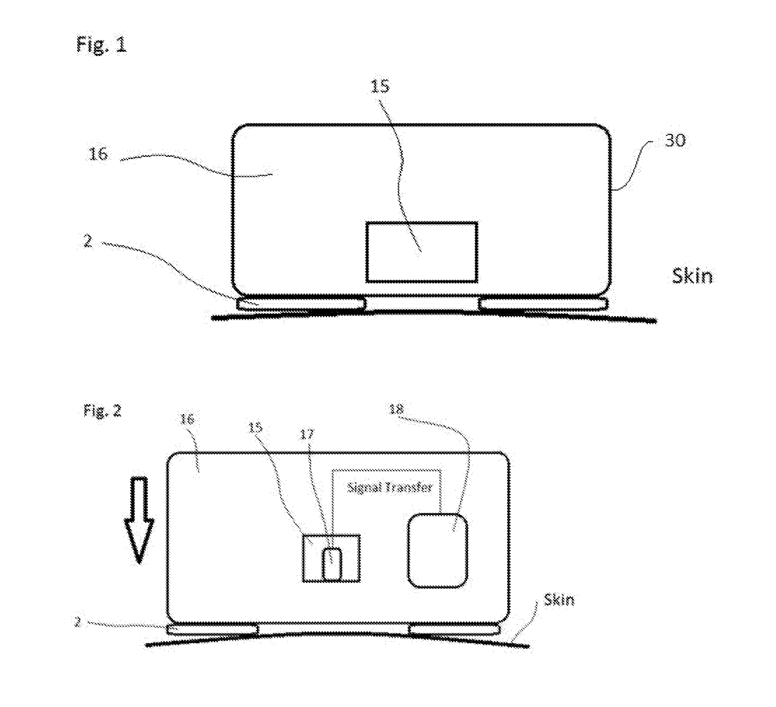

An example device 16 in accordance with the present disclosure is shown in FIG. 1. The device 16 is used to reduce a pain of skin invasion such as an injection, lancing, intubation, drawing blood, and tattooing. The device 16 includes a housing 30 that houses and/or supports various operating features and components and of the device 16. The device 16 can be positioned on the skin and apply electricity and physical stimulation to the skin on or in the vicinity of an injection site before and/or during an injection to reduce the pain of the injection. The device 16 includes a housing 30 that retains one or more stimulators 15 that physically stimulate the skin at least at the injection site. Physical stimulation can, for example, take the form of tapping, rubbing, and blowing gas among others. Examples of stimulators that can provide one or more of these modalities include among others, e.g., a rotating tapper than rotates around an axis and taps the skin on or around the injection site, a wheel rotating around an axis that is mainly perpendicular to the skin at the injection site, or a nozzle that blows compressed gas on or around the injection site. The device 16 may further include one or more electrodes 2 having two sides. The one or more electrodes are positioned so that one side of the electrode will come in electrical contact with the skin when in ready position. The side that comes in contact with the skin may include an electrolyte or an electrode gel that facilitates electrical transmission to the skin.

Physical stimulation to the skin is effected by one or more stimulators 15 that may be positioned at a distance from the injection site that makes it possible for the one or more stimulators 15 to effectively stimulate the skin. When physical stimulation such as tapping, rubbing, or blowing gas is applied, it is especially important that the stimulator(s) 15 is/are placed at a preset or predefined distance from the injection site and its adjacent areas to bring about effective stimulation, or within a predefined range of distances from the injection site and its adjacent areas. If the stimulator(s) 15 is/are too close or too far from the injection site, physical stimulation may be adversely affected. It is possible to use only one modality or more than one modality of physical stimulation at one time. A stimulator 15 with one modality of physical stimulation may require a different distance or range of distances from the skin than a stimulator 15 with another modality of physical stimulation in order to operate effectively. Similarly, the parameters of a particular modality or combination of modalities of physical stimulation may be modulated in a way that affects the optimal distance or range of distances between a particular stimulator 15 and the injection site.

In some examples, the device 16 includes a sensor 17 (see FIG. 2) supported by the housing 30. The sensor 17 is adapted to detect a distance between the stimulator 15 and the injection site and determine if the detected distance is within or without a predefined acceptable operating for the device 16. The user presses the device 16 towards the skin. As the device 16 moves towards the skin, the sensor 17 gages the distance between the stimulator 15 and the skin and signals the processor 18 when the distance is in an acceptable range. The processor 18, for example, compares the signal received from the sensor 17 with a look-up table stored on a memory to detect when the stimulator 15 is in an acceptable range.

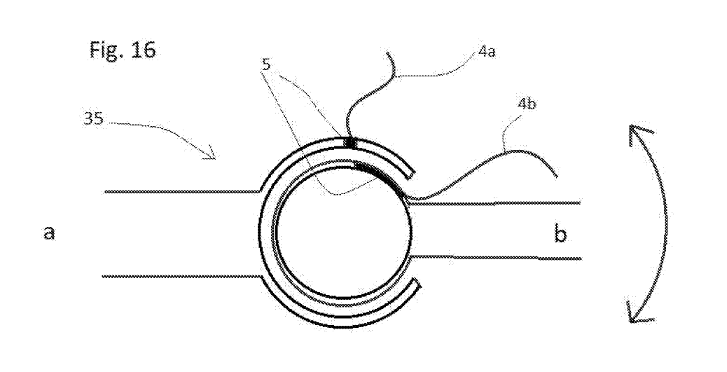

FIG. 5 shows two electrodes 2 that are flexible and springy in nature. When the device 16 is pressed on the skin, the electrodes 2 flex and adapt to the contour of the skin while the stimulator 15 comes closer to the skin and reaches a preset calculated range of distances from the injection site 40. In another example, the two electrodes are each held by a hinge 35 to the device 16 and can rotate around the hinges and better adapt to the contour of the skin. In an example, at least one hinge 35 has two general components, component a and component b, with component a fixed to the rigid part of the housing 30, and component b bearing a rotating electrode 2, as in FIG. 16. A conductive strip 5 is positioned on each component a and b so that at certain angles during the rotation of the component b over component a, the conductive strips slide over each other, during which time electricity can pass from wire 4a to wire 4b. The hinge 35 acts as a sensor and senses the angle degrees that the electrode 2 opens. When the electrode 2 opens to a predefined range of degrees, the hinge 35 sends a signal that can alert the user, that a processor 18 uses to activate the stimulator 15 and/or the electrodes 2 that provide electric stimulation, or that is used to directly activate the stimulator 15 and/or the electrodes 2. One or more electrodes 2 can be spring loaded to keep the electrodes in un-flexed/un-rotated position when the device 16 is idle.

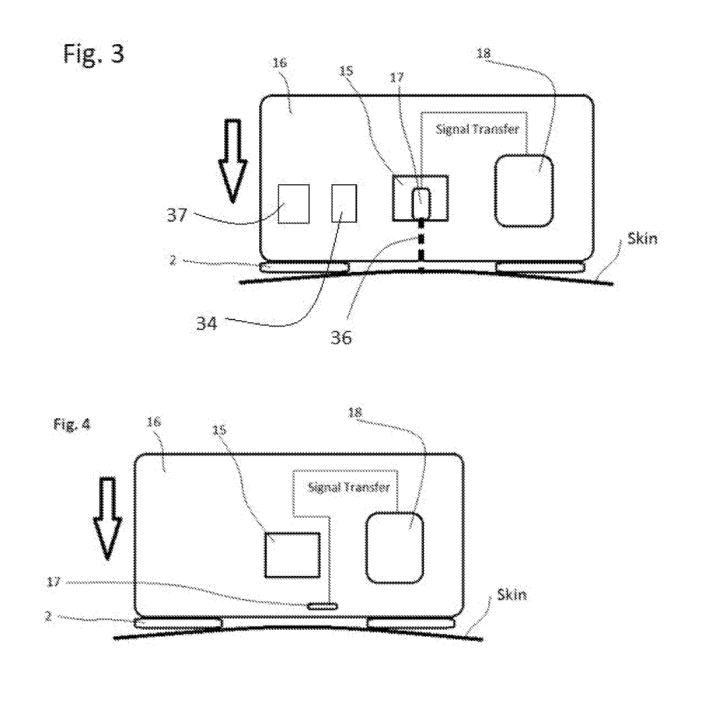

In one example, a signal corresponding to a detected distance between the stimulator 15 and the injection site is provided by the sensor 17 to a processor 18, FIG. 3. The processor 18, which can be adapted to execute computer-readable instructions stored in a non-transitory computer readable medium (such as a memory 37), can process the received signal to alert the user to stop pressing the device 16 on the skin any further if, for example, the detected distance is smaller than a minimum acceptable distance. In another example, e.g., if the detected distance is within a predefined operating range, the processor 18 processes the signal from the sensor 17 and causes the one or more stimulators 15 to start stimulating the skin. In another example, the processor 18 processes the signal from the sensor 17 and initiates application of electricity to the skin by one or more electrodes 2. In another example, the sensor 17 routinely generates signals corresponding to a detected distance between the stimulator 15 and the injection site, but stops providing distance signals when one or more of the stimulators 15 included in the device 16 is/are within an acceptable operating distance range from the injection site.

In one or more of the foregoing examples, the processor 18 can be coupled to a controller 34, the controller 34 being adapted to control activation and deactivation (e.g., by generating electronic control signals) of the one or more stimulators 15.

The sensor 17 may use one or more of a variety of mechanisms to gage the distance between a stimulator 15 and the injection site. For example, as shown in FIG. 3, the device 16 may use a sensor 17 that generates laser 36 to measure the distance between the stimulator 15 and the injection site 40. Alternatively, the sensor 17 can emit sound waves and measure the time it takes for the echo to bounce from the injection site or its vicinity back to the sensor. In another example, as shown in FIG. 4, the sensor 17 is so positioned on the housing 30 as to come in contact with the skin in close proximity to the injection site when the device 16 is pressed against and moves towards the skin. When the sensor 17 comes in contact with the skin, the sensor 17 alerts the user or signals the processor that a stimulator 15 is within an acceptable distance range from the injection site. In another example, when the sensor 17 comes in contact with the skin, it initiates electrical transmission via the one or more electrodes 2 and/or operation of the stimulator 15.

In an example of the devices of the present disclosure, sensor 17 is attached to a rigid part of the housing 30, is physically movable with respect to the housing 30, and can be moved by the rising or descending level of the skin at or in the vicinity of the injection site 40, FIG. 13. FIG. 13 shows the sensor 17 to be mounted on a hinge 35 that is attached to the rigid part of the housing 35. When the device 16, along with the housing 35 is pressed towards the skin, the tissue bulges under the sensor 17 and presses the sensor 17 away from the skin. The sensor 17 then moves away from the skin. Due to the rigidity of the housing 30, the distance between the stimulator 15 mounted thereon to the point where sensor 17 joins the rigid housing 30 is known. When the level of the skin at or in the vicinity of the injection site 40 varies with respect to the housing 30, for example by pressing the device 16 to the skin and causing the skin to bulge, the level of the sensor 17 that moves with the level of the skin also varies in tandem. By measuring the position of the sensor 17 with respect to its joint point with the rigid part of the housing 30, one can accurately estimate the distance between the stimulator 15 and the injection site. One example of measuring the position of the sensor 17 with respect to its joint point with the rigid part of the housing 30 is by gaging the angle of the sensor 17 to the rigid part of housing 30. The hinge 35 can simply have an on-off switch that turns on the stimulator 15 and the electrodes 2 when the angle is in an acceptable range. Alternatively, the hinge 35 can send signals to a controller 34 when the angle is in an acceptable range. Or the hinge 35 can alert the user that the angle is in an acceptable range. In a related example of the disclosed device, at least one electrode 2 or part thereof acts as a sensor 17 when for example the at least one electrode 2 or part thereof is placed in close proximity to or on the injection site.

In an example of the disclosed device, as shown in FIG. 5, at least a portion of an electrode 2 of the device 16 is resiliently flexible. As the device 16 is pressed on the recipient's skin and moving towards the injection site, the flexible portion of the electrode 2 flexes and/or rotates around an axis in order to stay relatively stationary with respect to the skin around the injection site. A sensor 17 gages the distance of the flexible portion of the electrode 2 with respect to the stimulator 15 and signals when it is in an acceptable range. In a preferred example of the devices of the present disclosure, the force needed to flex the electrodes to conform to the contour of the skin is less than the force needed to invaginate the skin 1 mm. In some examples, the flexibility of the electrodes 2 is such that the force needed to flex the electrodes 2 to conform to the contour of the skin is less than 0.1 N.

As shown in FIG. 5, in an example device according to the present disclosure, one or more electrodes 2 are each held by a support 19 extending from the housing 30 of the device 16. For example, the one or more electrodes 2 may be flexible; or in another example, one or more electrodes 2 may be springy. When the device 16 is pressed against the skin of the injection recipient, the one or more electrodes 2 flex and better adapt to the contour of the skin so as to improve the supply of electricity to the skin.

In another example, one or more electrodes 2 are rigidly held to the housing 30, FIG. 2. When the device 16 is pressed against the skin, the one or more electrodes 2 also press against the skin causing the skin to bulge around the electrodes 2. A sensor 17 gages the distance of the bulging skin with respect to a stimulator 15 and signals when the skin is in a preset range of distance from the stimulator 15. That signal can be used to alert the user to stop pressing further and/or to cause the stimulator 15 to physically stimulate the skin. A signal from a sensor 17 may also cause the one or more electrodes 2 to apply electricity to the skin.

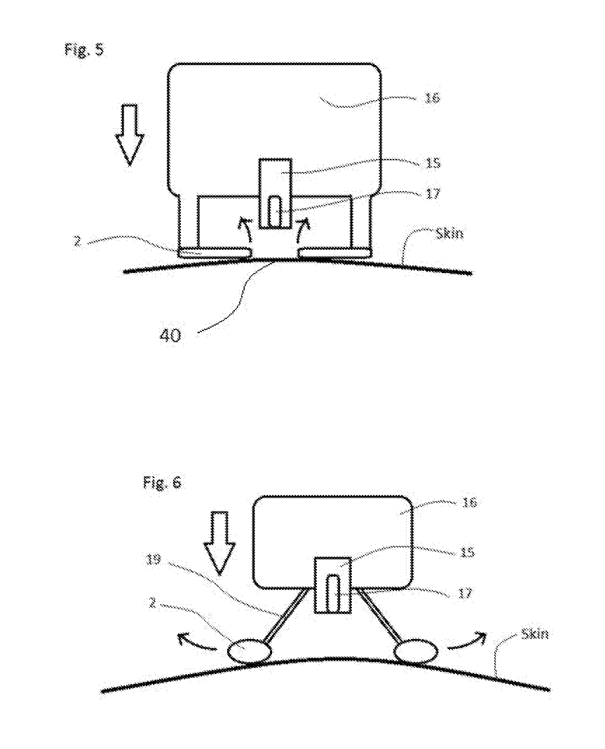

In a different example, as shown in FIG. 6, one or more electrodes 2 are each held to the device 16 by at least one support 19 extending from the housing 30 of the device 16. Each of one or more supports 19 can be rigid or resiliently flexible. When the device 16 is pressed against the skin, at least one of the one or more supports 19 move or flex to the side and allow the stimulator 15 to draw closer to the skin. For example, the supports 19 can be mounted on the device 16 and rotate around an axis so that the supports 19 move to the side and allow the device 16 to approach the skin.

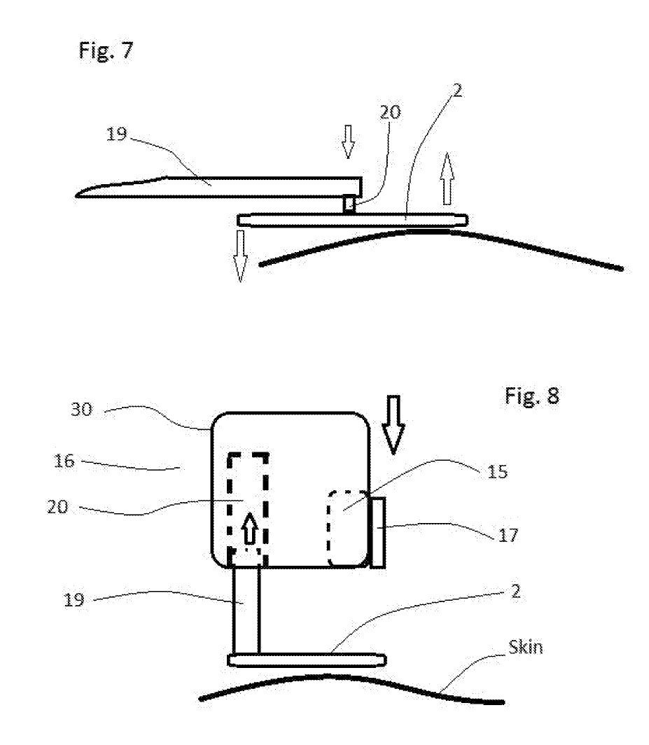

Referring to FIG. 7, in one example the one or more electrodes 2 are held by the one or more supports 19 through one or more swivels 20. A swivel 20 allows the electrode 2 some play with respect to the support 19 and helps the electrode 2 to better adapt to the contour of the skin, FIG. 7.

In a further example of a device of the present disclosure, as shown in FIG. 8, a support 19 that supports an electrode 2 is movably coupled to the housing 30. For example, the support 19 can travel back-and-forth inside a channel 20, provided in housing 30, that allows the electrode 2 to get closer to or farther from the housing 30. A spring inside the channel 20 exerts a force on the support 19 to push it out of the channel 20. When the housing 30 is pressed towards the skin, the spring in the channel 20 forces the support 19 outwardly towards the skin, which in turn pushes and presses one or more electrodes 2 onto the skin. Due to the force exerted on the one or more electrodes 2, the one or more electrodes 2 adapt to the contour of the skin. If the housing 30 is pressed further towards the skin, the skin reciprocates the force back to the one or more electrodes 2, keeping the one or more electrodes 2 relatively stationary while the housing 30 keeps moving towards the skin, in effect causing the supporting carrier 19 to move inside channel provided in the housing 30.

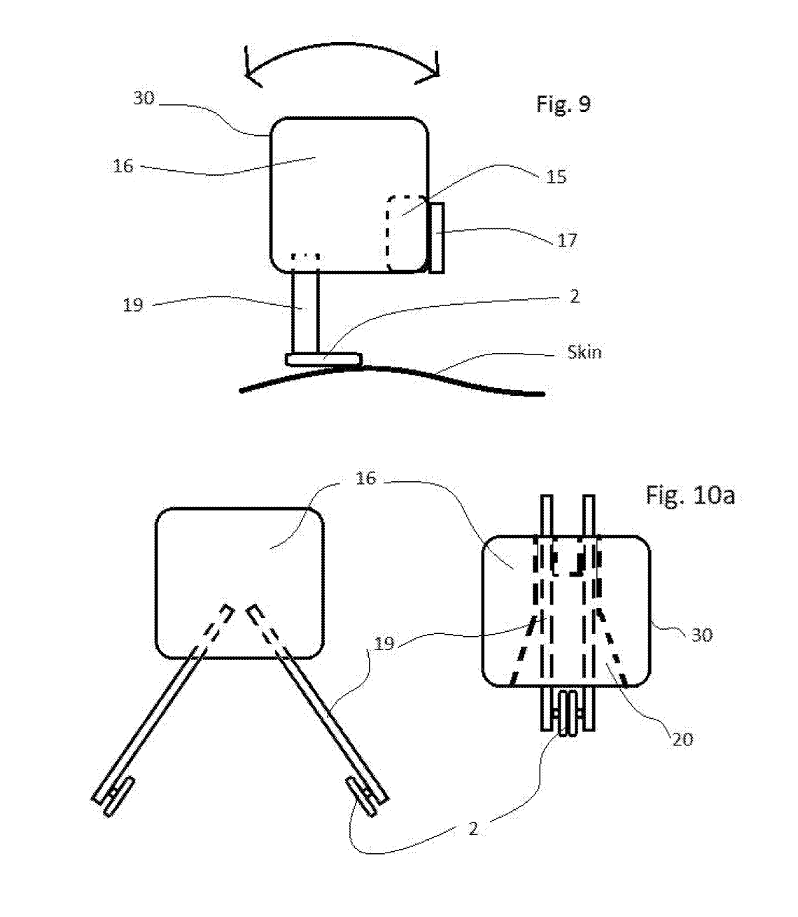

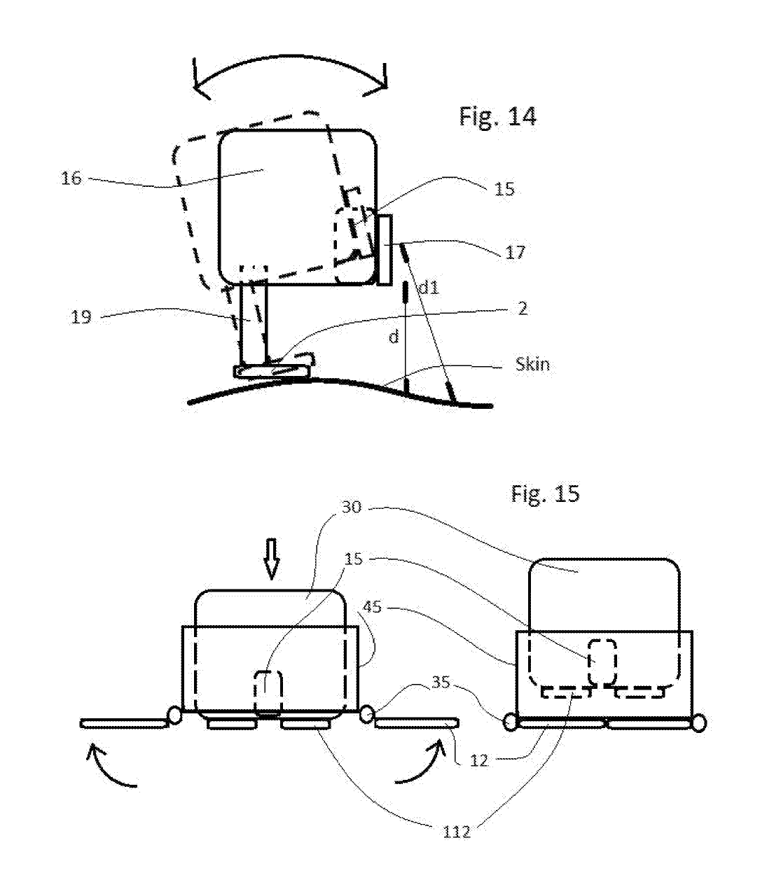

As FIG. 9 Shows, in a further example of a device in accordance with the present disclosure, the housing 30 includes one or more electrodes 2 touching the skin. The housing 30 can be tilted relative to the skin, e.g., in either direction represented by the arrow 60, see FIG. 14, so as to vary the distance d (see FIG. 14) between the included stimulator 15 and the skin. In FIG. 14, the distance between the stimulator 15 and the skin is shown as d when the device 16 is resting on the skin. When the device 16 is tilted, the distance between the stimulator 15 and the skin is represented as dl which is different than d depending on the direction and the extent of the tilt.

In an alternative variation of the example, the device 16 does not include a sensor 17. A skilled user tilts the device 16 until the user senses adequate physical stimulation on the skin. The user then keeps the device 16 in that angulation on the skin until the skin is properly anesthetized. For example, after the user places the device 16 on the skin, he may tilt it 10, 15, 20, 25, 30 or any other degrees to obtain a proper distance d between the stimulator and the injection that effectively causes anesthesia.

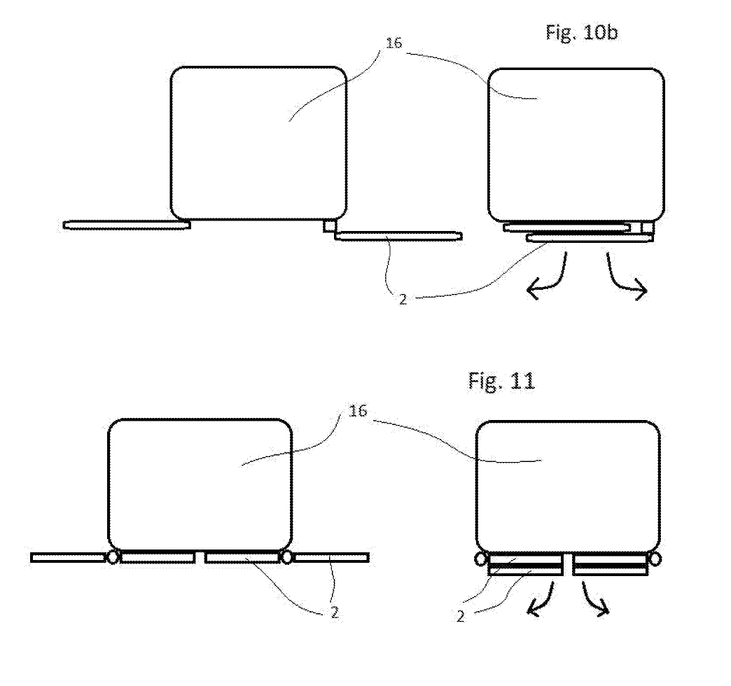

In order to better conduct electricity to the skin, the one or more electrodes 2 can be coated with an electrically conducting solution (electrolyte) such as saline or an electrode gel. These solutions sometimes dry over time and lose their conductivity. In order to prevent these solutions from drying, two or more electrodes 2 are brought together or an electrode 2 is folded over itself or another surface when not in use to avoid the solution liquid evaporation. For example, as shown in FIG. 10a, a device 16 includes two supports 19, each holding an electrode 2. At least one of the supports 19 is adapted to move with respect to the other support 19 so as to bring together or to separate the held electrodes 2.

In another example, one or more supports 19 are rigid and rotate around an axis as in FIG. 6. When the device 16 is pressed towards the skin, the one or more supports 19 rotate outwardly allowing the device 16 to approach the skin. In another example, at least part of at least one of the supports 19 is resiliently flexible so as to enable to bring together or to separate the held electrodes 2. When the device 16 is pressed towards the skin, the supports 16 flex outwardly and allow the device 16 to approach the skin.

In another example, at least one of the electrodes 2 is resiliently flexible. When the electrodes 2 are pressed against the skin, they flex and adapt to the skin. When they disengage the skin, they flex back to a relatively straight shape.

FIG. 10a shows two supports 19, each carrying an electrode 2. The supports 19 can move up and down inside a channel 20 provided inside the housing 30. When not in use, the supports 19 can be stowed up the channel 20 so that the electrodes 2 come in contact with each other. This arrangement prevents drying of any electrolyte that may be placed on the electrodes 2. Once they are required to apply electricity, they are extruded out of the channel 20 allowing the electrodes 2 to contact the skin.

FIG. 10b shows a device 16 including two electrodes 2. The electrodes 2 can fold on each other, when the device 16 is not in use, so as to preserve any electrolyte that is on them. Before operation, the user can flap open the electrodes and expose the electrolyte on them.

FIG. 11 shows a device 16 that can have two or four electrodes 2. If the device has two electrodes 2, then each electrode 2 folds on itself when the device 16 is not in use. If the device has four electrodes 2, then two of the electrodes flap closed so as to face and contact the other two electrodes of four when the device 16 is idle. Before operation, the user can unfold the electrodes and expose the electrolyte on them.

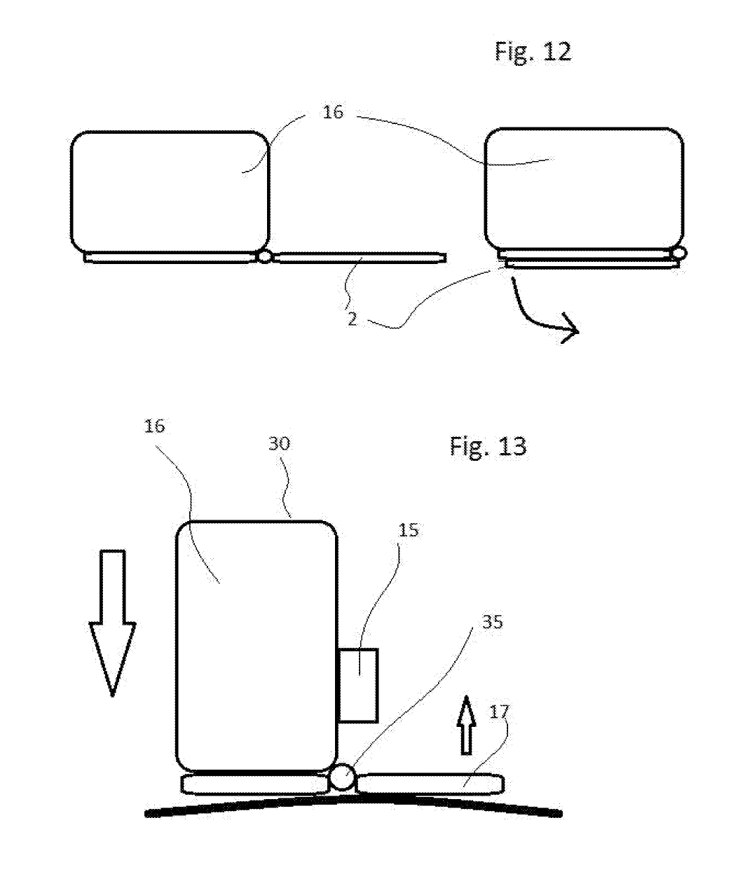

FIG. 12 similarly shows two electrodes 2 included in a device 16. One electrode 2 is stationary on the device 16, whereas the second electrode 2 can face and contact the first electrode 2 when the device 16 is not in use, and can also flap open when the device 16 is intended to be used.

In a preferred example of the invention, at least part of at least one electrode 2 moves in a range of motion. In one point in the range of motion, which could be an endpoint, at least part of the electrode 2 reaches another surface. If there is an electrolyte applied to the electrode, this contact with the other surface helps prevent drying of the electrolyte, for example, when the device 16 holding the electrode 2 is not in use. At another point in the range of motion of the moving part of the electrode 2, which could be the other endpoint of the range of motion, the moving part of electrode 2 rests on the skin and brings at least one stimulator 15 to a proper distance from the injection site in order for the stimulator to adequately stimulate the injection site. The other surface that comes in contact with the electrolyte on the electrode 2 can for example be another electrode 2, another part of the same electrode 2, or part of the housing 30, as illustrated in FIGS. 10a, 10b, 11, and 12. Part of the electrode 2 can move in a range of movement because, for example, at least part of the electrode 2 is flexible and allows folding or flexing part of the electrode 2 on a different surface, at least part of the electrode 2 can rotate around a hinge, or at least part of the electrode 2 is transported by a support 19.

FIG. 10b shows 2 electrodes 2 that fold over each other when not in use to take less space, to prevent drying of electrolytes on them, and to keeps the stimulator 15 inside the device 16 sterile. When intended to be used, the electrodes 2 flip open and expose their side with electrolytes that then can contact the skin

In FIG. 15, an embodiment of the present device 16 is exemplified showing the housing 30 at least partially included inside a conduit 45. The housing 30, having at least one stimulator 15, is movable inside the conduit 45. The conduit 45 has at least one opening. At least one opening in conduit 45 is protected by at least one flap 12 that closes the opening when the device 16 is not intended for use in the near future. When a user intends to use the device 16, the at least one flap 12 opens. For example, the housing 30 moves at least partially out of the conduit 45 so that the stimulator 15 can physically stimulate the injection site. In an example, one or more electrodes 112 are included on the housing 30. In another example, the at least one flap 12 is an electrode. In an example, the electrodes 12 and/or 112 have a layer of electrolyte on their surface that will be exposed to the skin. In a preferred example of the embodiment, moving the housing 30 towards the at least one flap 12 causes the at least one flap 12 to flap open. For example, the housing 30 physically pushes the flap 12 open. This feature of the embodiment covering the stimulator 15 and electrodes 12 and/or 112 prevents the stimulator from contamination and the electrolyte from drying. In an example, at least one flap 12 is mounted on a hinge 35 to facilitate opening and closing of the flap 12. In an example, at least one hinge 35 acts as a sensor and senses the angle degrees that the flap 12 opens. When the flap 12 opens to a predefined range of degrees, the hinge 35 sends a signal that can alert the user, that a processor 18 uses to activate the stimulator 15 and/or the electrodes 2, 12, and/or 112 that provide electric stimulation, or that is used to directly activate the stimulator 15 and/or the electrodes 2, 12, and/or 112. One or more flaps 12 can be spring loaded to keep the flap closed when the device 16 is idle.

One skilled in the art will appreciate that the present invention can be practiced by other than the described embodiments, which are presented here for purposes of illustration and not of limitation, and the present invention is limited only by the claims that follow.

What is claimed is:

1. A method of reducing an injection pain by using a device positioned on an injection-recipient skin near an injection site, said device including a) at least one stimulator positioned on said device at a distance from the skin in order to physically stimulate the skin on or near the injection site, b) one or more electrodes that can apply electricity to the skin to the injection site, and c) a sensor that can gage the distance of said at least one stimulator to said injection site or an area of the skin adjacent to said injection site; said method comprising placing said device on the skin and pressing it to the skin until said at least one stimulator reaches a desired distance from said injection site which is gaged by said sensor which then generates a signal.

2. The method according to claim 1 wherein said sensor uses laser to gage the distance between said one or more stimulators and said injection site or the area of the skin adjacent to said injection site.

3. The method according to claim 1 wherein said sensor is positioned a distance away from said injection site and sends a signal when said sensor touches the skin near the injection site.

4. The method according to claim 1 wherein said signal is used to alert the user of said device to stop pressing said device further into the skin.

5. The method according to claim 1 wherein said signal prompts said at least one stimulator to physically stimulate the skin.

6. The method according to claim 1 wherein said signal prompts said one or more electrodes to apply electricity to the skin.

7. The method according to claim 1 wherein said one or more electrodes are rigidly held to said device.

8. The method according to claim 1 wherein at least one of said one or more electrodes is held by one or more supports that flex when said device is pressed against the skin, allowing said at least one stimulator to approach the skin.

9. A device for reducing pain of an injection, comprising a housing; at least one stimulator supported by the housing and positioned on said housing such that the stimulator is adapted to be placed at a distance from a skin of a patient; one or more electrodes supported by the housing and adapted to apply electricity to the skin; and a sensor supported by the housing, the sensor being adapted to measure the distance between the stimulator and the skin of the patient.

10. The device according to claim 9 wherein said one or more electrodes are rigidly held by said housing.

11. The device according to claim 9 wherein at least one of said one or more electrodes are held by at least one or more supports.

12. The device according to claim 9, wherein said sensor comprises a laser.

13. The device according to claim 9, wherein at least one of said electrodes is held by a hinge that is fastened to a rigid part of said housing, said hinge sending a signal when the said at least one held electrode is at certain predefined range of angles with respect to said housing.

14. The device according to claim 9, wherein said signal is processed by a processor, and the processor causes an alert to be generated, the alert being adapted to alert a user of the device to stop pressing said housing further towards the skin.

15. The device according to claim 9, wherein said signal is processed by a processor, and the processor causes said at least one stimulator to physically stimulate the skin.

16. The device according to claim 9 wherein said signal is processed by a processor, and the processor causes said at said one or more electrodes to apply electricity to the skin.

17. A device to reduce the pain of an injection comprising a) at least one stimulator to physically stimulate the skin at or around the injection site, and b) one or more electrodes that can apply electricity to the skin to the injection site, wherein at least part of at least one said electrode is movable in a range of movement, said range of movement including at least one point at which at least part of at least one of said electrodes contacts a surface and at least another point at which at least part of at least one of said electrodes is so positioned as to bring said stimulator on said device to a distance from the injection site in order to effectively physically stimulate the skin on or near the injection site.
